(19) United States
(12) Patent Application Publication
(10) Pub. No.: US 2018/0268569 A1
(43) Pub. Date: Sep. 20, 2018

(54) METHOD AND MEDICAL IMAGING APPARATUS FOR DETECTING ABNORMALITIES IN MEDICAL IMAGE DATA OF A REGION OF A PATIENT OUTSIDE OF A REGION TO BE EXAMINED

(71) Applicant: Siemens Healthcare GmbH, Erlangen (DE)

(72) Inventor: Maria Kroell, Erlangen (DE)

(73) Assignee: Siemens Healthcare GmbH, Erlangen (DE)

(21) Appl. No.: 15/921,054

(22) Filed: Mar. 14, 2018

(30) Foreign Application Priority Data
Mar. 14, 2017 (DE) 102017204175.7

(51) Int. Cl. G06T 7/90; G06T 7/00; G06T 7/11; G06T 7/174; A61B 5/00

(52) U.S. Cl. CPC G06T 7/90; G06T 7/0014; G06T 7/11; G06T 7/174; A61B 5/4842; A61B 5/743; G06T 2207/30004; A61B 5/7267; G06T 2207/10081; G06T 2207/10104; G06T 2207/10088; G06T 2207/20081; A61B 5/7485

(57) ABSTRACT

In a method and medical imaging apparatus for detecting abnormalities in medical image data of a region of the patient that is outside of a region to be examined of the patient, medical image data that depict a region of the patient that is outside of the region to be examined of the patient are provided in a computer, wherein the region to be examined of the patient has already been selected on the basis of preliminary examination data. The computer automatically evaluates the medical image data for the region that is outside of the region to be examined of the patient, and generates abnormality information of the medical image data for the region that is outside of the region to be examined of the patient. The abnormality information is visually presented.

### BACKGROUND OF THE INVENTION

### Field of the Invention

The present invention concerns a method for detecting abnormalities in medical image data of a region of the patient that is outside of a region to be examined of the patient. The present invention further concerns a medical imaging apparatus having a raw data acquisition scanner, a control computer and a display, the medical imaging apparatus being designed to perform such method. The present invention also concerns a medical imaging apparatus and a non-transitory, computer-readable data storage medium designed to implement such a method.

### Description of the Prior Art

Medical imaging examinations are often carried out under time pressure. This means the planning, execution and evaluation of the medical imaging examination are usually limited to and/or focused on solely the region of the patient that is to be examined. For example, if the region of the patient that is to be examined is the kidney of the patient, then the planning, execution and evaluation of the medical image data are limited to and/or focused and/or concentrated on the acquisition of raw data that are to reconstruct into image data that depicts the kidney of the patient.

Abnormalities in the medical image data that depict regions of the patient that are outside of the region to be examined of the patient are therefore not detected, and are not clinically assessed.

### SUMMARY OF THE INVENTION

An object of the present invention is to identify abnormalities in medical image data of regions of the patient that are outside of the region to be examined.

A method according to the invention for detecting abnormalities in medical image data of a region of the patient that is outside of a region of the patient that is to be examined has the following steps.

Medical image data that depict a region of the patient that is outside of the region to be examined of the patient are provided to a computer, wherein the region to be examined of the patient has already been selected on the basis of preliminary examination data.

The computer automatically evaluates the medical image data for the region that is outside of the region to be examined of the patient.

Abnormality information about the medical image data for the region that is outside of the region to be examined of the patient automatically generated by the control computer.

The abnormality information is shown at a display screen in communication with the computer.

As used herein, abnormalities in medical image data mean abnormalities in evaluated medical image data, the abnormalities being identified by virtue of a change in color and/or a change in contrast in the evaluated medical image data, wherein the change in color and/or change in contrast is not caused by anatomy of the patient. Subregions containing the abnormalities in the evaluated medical image data stand out by virtue of a change in contrast and/or by virtue of a change in color from subregions that image an environment of the abnormalities. The abnormality in the medical image data may also be a deviation in the medical image data from a normal state. For example, a dark spot within an imaged organ of the patient may represent an abnormality of this kind.

The medical image data preferably are medical imaging data reconstructed from raw data acquired by a medical imaging apparatus. The medical imaging apparatus may be, for example, a computed tomography apparatus, a positron emission tomography (PET) apparatus, a magnetic resonance apparatus, etc. The medical image data may accordingly be computed tomography image data, PET image data, magnetic resonance image data, etc.

The region to be examined of the patient is a locally delimited region within the patient. For example, the region to be examined or the locally delimited region can be an organ or a joint region of the patient. In a diagnostic imaging examination of the region to be examined of the patient, raw data are acquired from the region to be examined of the patient and image data are reconstructed therefrom.

The region of the patient that is to be searched and/or screened with respect to an abnormality is not encompassed by the region to be examined, i.e., it is outside of the region to be examined of the patient. For example, if the region to be examined of the patient is an organ, then the region that is located around the organ can be searched and/or screened with respect to an abnormality in the medical image data. The region that is screened and/or searched with respect to an abnormality in the medical image data may in this case be directly adjacent to the region to be examined of the patient. Furthermore, the region that is screened and/or searched with respect to an abnormality in the medical image data may be spaced apart at a distance from the region to be examined within the patient.

Providing medical image data that depict a region of the patient that is outside of the region to be examined of the patient may preferably comprise an acquisition of medical image data, result from an acquisition of raw data in an overview measurement and/or a localizer measurement. An overview measurement and/or localizer measurement is produced of the region of the patient that is outside of the region to be examined of the patient, and therefore is not encompassed by the region to be examined of the patient, and that is intended to be searched and/or screened with respect to an abnormality. In this case, the overview measurement or the localizer measurement of the region of the patient that is outside of the region to be examined of the patient typically has a lower resolution, in particular a lower spatial resolution, than the medical diagnostic image data of the region to be examined of the patient.

Furthermore, providing medical image data that depict a region of the patient that is outside of the region to be examined of the patient may also be combined with an acquisition of diagnostic image data that are acquired for the purpose of resolving diagnostic issues pertaining to the region to be examined of the patient. For example, the region to be examined of the patient may be a kidney of the patient. The kidney of the patient is therefore depicted in the acquired diagnostic image data and, for example, a region that is outside of the region to be examined of the patient is also imaged in a border region of the acquired diagnostic image data. This border region, for example, may depict a further organ of the patient, such as the liver of the patient. This border region may therefore be screened and/or searched with respect to an abnormality.

The region to be examined of the patient preferably has been selected on the basis of preliminary examination data of a preliminary examination. During the preliminary examination, a diagnostic issue with respect to the region to be examined of the patient is in question, which is intended to be resolved by the medical imaging examination of the patient.

The automatic evaluation of the medical image data for the region that is outside of the region to be examined of the patient is carried out by the control computer of the medical imaging examination. To that end, the control computer has evaluation programs and/or evaluation software that are/is stored in a memory unit and are/is executed by a processor of the control computer. In this case, the memory may be incorporated in the control computer and/or the medical imaging apparatus. The memory may also be an external storage source, such as a storage source in a cloud, etc. The medical image data of the region that is outside of the region to be examined is evaluated with respect to an abnormality and/or a deviation by the evaluation programs and/or the evaluation software.

The abnormality information is generated automatically and/or autonomously by the control computer of the medical imaging apparatus.

The abnormality information preferably includes information as to whether an abnormality has been detected or identified in the medical image data for the evaluated medical image data of the region that is outside of the region to be examined. Moreover, the abnormality information may also include an alert indicating that further medical imaging examinations are required for a possible clinical assessment of the region of the patient that is outside of the region to be examined of the patient. The abnormality information is generated by the control computer on the basis of the evaluated medical image data.

The invention has the advantage that a member of the medical operating staff supervising the medical imaging examination is directly and automatically alerted to possible abnormalities in the image data of the patient. This enables errors in the assessment of the image data to be reduced and/or avoided, since not only the diagnostic image data of the region to be examined are taken into consideration in the assessment, but also the assessment can be based on the abnormality information.

In an embodiment of the invention, providing the medical image data includes an acquisition of medical image data of the region of the patient, the region being outside of the region to be examined of the patient. Medical image data of the region of the patient that is outside of the region to be examined of the patient is preferably acquired by an overview measurement or a localizer measurement. This has the advantage that the medical image data for detecting abnormalities can be provided particularly quickly and as a result virtually no delays occur in examination workflow. The patient too perceives the measurement time during which he or she is situated within the patient receiving zone as not significantly longer, such that any discomfort experienced by the patient is not exacerbated.

In a further embodiment, the medical image data of the region of the patient that is outside of the region to be examined of the patient are acquired during a diagnostic imaging examination, for acquiring diagnostic image data of the region to be examined of the patient. Preferably, medical image data of the region that is outside of the region to be examined of the patient are acquired during the diagnostic imaging examination, such as during a measurement pause between two diagnostic imaging measurements, for example, or during a planning phase for setting measurement parameters for a pending diagnostic imaging measurement of the region to be examined. Diagnostic imaging data of the region to be examined of the patient are acquired by the diagnostic imaging examination. This embodiment of the invention has the advantage that the total duration of the imaging examination of the patient, during which diagnostic image data of the region to be examined of the patient as well as medical image data of the region that is outside of the region to be examined of the patient, are acquired, does not have to be significantly extended. The result is that the acquisition of the medical image data of the region of the patient that is outside of the region to be examined can be performed in a particularly time-saving and expeditious manner. Thus, the length of time during which the patient resides or is present within a patient receiving zone of the medical imaging device is kept to a minimum.

Preferably, the medical imaging measurement for acquiring medical image data of the region that is outside of the region to be examined of the patient is planned and/or performed automatically by the control computer. In this case, the planning, and preferably also the execution of the medical imaging measurement for acquiring medical image data of the region that is outside of the region to be examined of the patient, are controlled automatically and/or autonomously by the control computer of the medical imaging apparatus. Preferably, even the initiation of the acquisition of the medical image data of the region that is outside of the region to be examined is effected automatically and/or autonomously by the control computer, such that there is no requirement for the medical operating staff either to plan or to perform the acquisition of the medical image data of the region that is outside of the region to be examined. This relieves the user, in particular the medical operator, of an additional workload required for the detection of abnormalities, while still enabling a result to be provided to the user concerning the presence of abnormalities.

In a further embodiment of the invention, the medical image data of the region of the patient are a whole-body scan of the patient, containing the region outside of the region to be examined of the patient. A whole-body scan or a whole-body acquisition means a medical imaging examination in which images and/or views of the entire body of the patient are acquired. This medical image data of the whole-body scan of the patient can also be acquired by an overview measurement and/or a localizer measurement. Preferably, medical image data for all regions of the body of the patient can be made available for the purpose of detecting abnormalities.

In another embodiment of the invention, the medical image data of the region of the patient outside of the region to be examined of the patient are inferior in terms of image quality to the image quality of diagnostic image data of the region to be examined of the patient. In this case, the medical image data of the region of the patient that is outside of the region to be examined of the patient have a lower resolution, in particular a lower spatial resolution, than the resolution, in particular a spatial resolution, of the diagnostic image data of the region to be examined of the patient. This permits a particularly short acquisition time for acquiring the medical image data of the region of the patient that is outside of the region to be examined of the patient.

The acquired medical image data of the region that is outside of the region to be examined of the patient are evaluated automatically by the control computer with respect to the presence of abnormalities. As a result, the acquired medical image data of the region that is outside of the region to be examined of the patient also does not require preprocessing in preparation for a human evaluation, which means that both the acquisition and the evaluation can be carried out in a particularly time-saving manner.

According to the invention, the automatic evaluation of the medical image data for the region that is outside of the region to be examined of the patient can be carried out by a self-learning algorithm incorporated in the control computer.

Typically, the self-learning algorithm is based on a machine learning technique in which knowledge is generated from experience. The machine learning is realized by artificial neural networks. By the machine learning process, the self-learning algorithm is able to recognize patterns and rules in learning data or training data, in particular assessed medical image data and the interpretation associated therewith. The self-learning algorithm can in this case learn from examples and generalize these following termination of the learning phase.

The self-learning algorithm or the machine learning may be based, for example, on a deep-learning method in which knowledge is generated from experience. In the deep-learning method, artificial neural networks are arranged in layers that use increasingly complex features in order, for example, to recognize the content of image data and/or to detect contrasts in image data. This enables large data resources to be classified into categories.

For this purpose, the control computer is configured with artificial intelligence that includes the self-learning algorithm. Preferably, the artificial intelligence involves methods that enable a computer to solve problems of a type that, when they are solved by human beings, require the use of intelligence resources. The computer may be configured by hardware or programs and software that allow problems to be processed independently by the computer. The artificial intelligence thus represents an automation of intelligent behavior. Preferably, the computer has the capability to learn and to deal with uncertainties and/or with probabilistic information.

With this embodiment of the invention, it is possible to perform a particularly time-saving evaluation of the medical image data of the region that is outside of the region to be examined with respect to a presence of abnormalities. Furthermore, a particularly reliable and efficient evaluation for detecting abnormalities in medical image data of a region that is outside of the region to be examined of the patient can be achieved in the process. Moreover, the evaluation can also be performed cost-effectively, since no additional investment of human resources is required.

Furthermore, the self-learning algorithm may be based on training data that is derived from assessed abnormalities in already-available findings of medical image data and/or diagnostic image data. Preferably, the already-available findings containing the assessed abnormalities are stored in a database, in which case the control computer can access the database, in particular the stored data of the database, via a data transmission unit. With the training data, the self-learning algorithm is able to learn to recognize a problem and/or detect an abnormality in the medical image data automatically and thus provide a reliable evaluation of the medical image data for an assessment by the medical operating staff.

In an embodiment of the invention, the self-learning algorithm takes into consideration data of a course of a disease and/or of preliminary examinations and/or further medical data of the patient in the evaluation of the medical image data. The further medical data of the patient may also include, for example, information relating to blood values and/or circulation values of the patient. A course of a disease of the patient may be a history of one or more disorders of the patient. Preliminary examinations may also include non-imaging preliminary examinations or even imaging examinations carried out using an imaging apparatus that is different from the current imaging apparatus. In this case, in addition to the currently acquired medical image data of the region that is outside of the region to be examined of the patient, further medically relevant data of the patient may also be taken into consideration in the evaluation of the medical image data. This also permits a targeted search for abnormalities in medical image data that images and/or visualizes defined and/or delimited regions of the patient, the defined and/or delimited regions being outside of the region to be examined. For example, if the preliminary examinations and/or a course of a disease and/or the further medical data point to a pulmonary disease of the patient, then a region in the medical image data that images the lung region of the patient can be focused on in the evaluation of the medical image data. During the evaluation of the medical image data, the self-learning algorithm may also take into consideration data that include already-acquired medical image data of the patient.

In another embodiment, the abnormality information is visualized or displayed in presented images of the diagnostic image data. This enables a good visibility of the abnormality information to be achieved for a member of the medical operating staff. The abnormality information can be displayed in this way directly to the medical operating staff during an assessment of the diagnostic image data.

The abnormality information preferably includes information for further assessment measures with respect to the abnormality, as a result of which a member of the medical operating staff can plan and/or carry out further examinations for assessing the abnormality in a simple and time-saving manner. Such further assessment measures may include, for example, suggestions for additional examinations of the region containing the abnormality. These additional examinations may also already include suggestions for further medical imaging examinations of the region containing the abnormality. A suggestion of this type may also include additional information, such as administration of contrast agent, for example, and/or parameter settings for a further medical imaging examination. A suggestion of this type may be confirmed, in particular accepted, by the user, such as a member of the medical operating staff, so further medical imaging examinations may also be performed immediately on the patient. Further medical imaging examinations may instead be performed on the patient at a later time if there are already waiting times for other patients for pending medical imaging examinations using the medical imaging apparatus. This enables further medical imaging examinations to be efficiently carried out.

The invention further concerns a medical imaging apparatus having an image data acquisition scanner, a control computer, and a display screen, the medical imaging apparatus being configured to perform the method according to the invention for detecting abnormalities in medical image data of a region of the patient that is outside of a region to be examined of the patient.

The medical imaging apparatus may be, for example, a computed tomography apparatus, a positron emission tomography (PET) apparatus, a magnetic resonance apparatus, etc. Accordingly, the medical image data may be computed tomography data, PET data, magnetic resonance data, etc.

The image data acquisition scanner thus can be a scanner of a computed tomography apparatus or a scanner of a PET apparatus or a scanner having a reception antenna for receiving magnetic resonance signals of a magnetic resonance apparatus, etc.

The invention has the advantage that a member of the medical operating staff supervising the medical imaging examination is directly and automatically alerted to possible abnormalities in the image data of the patient. This advantageously enables errors in an assessment of the image data to be avoided, since not only the diagnostic image data of the region to be examined are taken into consideration in the assessment, but the assessment can also be based on the abnormality information.

The advantages of the inventive medical imaging apparatus substantially correspond to the advantages of the inventive method for detecting abnormalities in medical image data of a region of the patient that is outside of a region to be examined of the patient, which advantages are explained in detail above. Features, advantages or alternative embodiments mentioned above are applicable to the apparatus as well.

The control computer can execute a self-learning algorithm that is provided for evaluating medical image data of a region of the patient that is outside of the region to be examined of the patient. This makes it possible to perform a particularly time-saving evaluation of the medical image data of the region that is outside of the region to be examined with respect to a presence of abnormalities. Furthermore, a particularly reliable and efficient evaluation for detecting abnormalities in medical image data of a region that is outside of the region to be examined of the patient can be achieved in the process. Moreover, the evaluation may be carried out particularly cost-effectively, since no additional investment of human resources is required.

The present invention also encompasses a non-transitory, computer-readable data storage medium encoded with programming instructions that, when the storage medium is loaded into a control computer of a medical imaging apparatus, cause the control computer to implement any or all embodiments of the method according to the invention, as described above.

### BRIEF DESCRIPTION OF THE DRAWINGS

FIG. 1 schematically illustrates a medical imaging apparatus according to the invention.

FIG. 2 is a flowchart of the method according to the invention for detecting abnormalities in medical image data of a region of the patient that is outside of a region to be examined of the patient.

### DESCRIPTION OF THE PREFERRED EMBODIMENTS

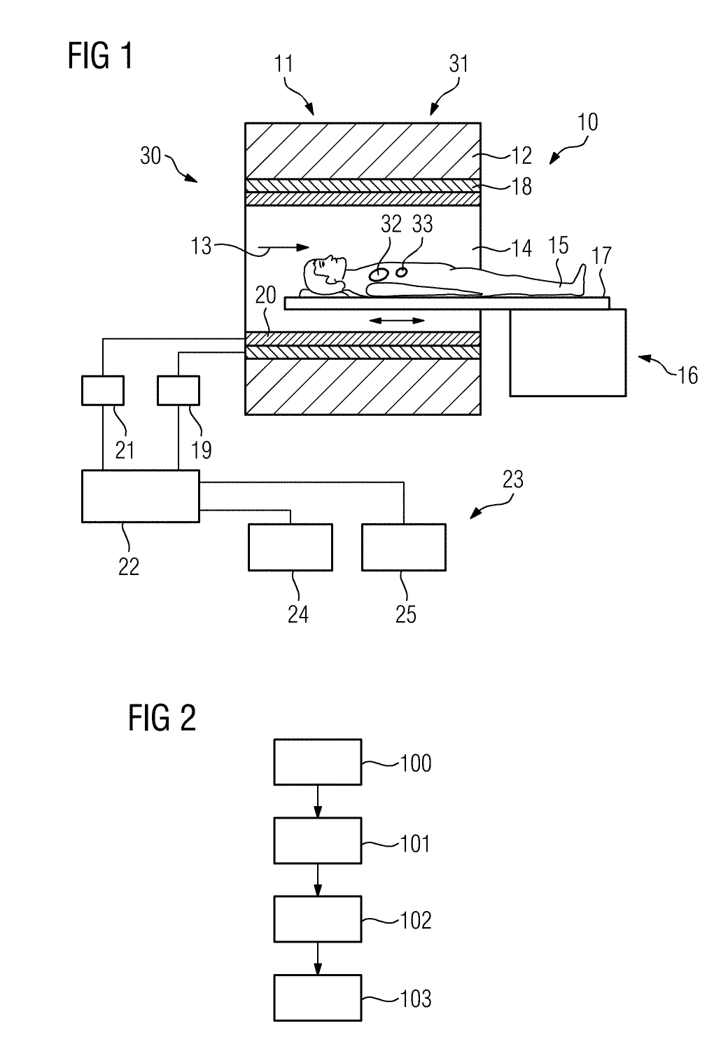

FIG. 1 schematically shows a medical imaging apparatus 30. In the exemplary embodiment, the medical imaging apparatus 30 is a magnetic resonance apparatus, the present invention being explained as an example with reference to the magnetic resonance apparatus. However, the present invention is not limited to an embodiment of the medical imaging apparatus 30 as a magnetic resonance apparatus. Other embodiments of the medical imaging apparatus 30 are conceivable, such as a computed tomography apparatus, a PET apparatus, etc.

The medical imaging apparatus 30 has an image data acquisition scanner 31. In the exemplary embodiment, the image data acquisition scanner 31 has a superconducting basic field magnet 12 that generates a strong and constant basic magnetic field 13. The scanner 31 has a patient receiving zone 14 for accommodating a patient 15. In the exemplary embodiment, the patient receiving zone 14 is embodied in the shape of a cylinder and is circumferentially enclosed by the scanner 31. In principle, however, a different embodiment of the patient receiving zone 14 is conceivable. The patient 15 can be introduced or moved into the patient receiving zone 14 by a patient support 16. For this purpose, the patient support 16 has a patient table 17, which is movable within the patient receiving zone 14.

The scanner 31 additionally has a gradient coil arrangement 18 for generating magnetic field gradients that are used for spatial encoding during an imaging session. The gradient coil arrangement 18 is controlled by a gradient controller 19. The scanner 31 further has a radio-frequency (RF) antenna 20 controlled by an RF antenna controller 21 so as to radiate RF sequences into an examination volume that is substantially formed by the patient receiving zone 14 of the scanner 31. The radiated RF sequence gives certain nuclear spins in the patient 15 a magnetization, which causes those nuclear spins to be deflected from the polarization produced by the basic magnetic field 13. As those excited nuclear spins relax and return to the steady state, they emit RF signals (MR signals) that are detected by the same antenna that radiated the RF sequence, or by a different RF antenna.

The magnetic resonance apparatus has a control computer 22 that controls the basic field magnet 12, the gradient controller 19 and the RF antenna control unit 21. The control computer 22 is responsible for the centralized control of the magnetic resonance apparatus, such as for performing a predetermined imaging gradient echo sequence, for example.

The magnetic resonance apparatus further has a user interface 23 connected to the control computer 22. Control information, such as imaging parameters, as well as reconstructed magnetic resonance images, can be displayed on an output unit 24, for example on at least one monitor, of the user interface 23 for a member of the medical operating staff. The user interface 23 also has an input unit 25 via which information and/or parameters can be entered by the medical operating staff during a measurement procedure.

FIG. 2 illustrates the inventive method for detecting abnormalities in medical image data, in particular magnetic resonance image data, of a region 32 that is outside of a region 33 to be examined of the patient 15. The magnetic resonance apparatus, in particular the control computer 22 thereof, is configured to perform and/or control the method for detecting abnormalities in medical image data, in particular magnetic resonance image data, of the region 32 that is outside of the region 33 to be examined of the patient 15.

To that end, the control computer 22 has computer programs and/or software that can be loaded directly into a memory, having program code for performing the method for detecting abnormalities in medical image data, in particular magnetic resonance image data, of the region 33 that is outside of the region 32 to be examined of the patient 15 when the computer programs and/or software are executed in the control computer 22. For this purpose, the control computer 22 has a processor (not shown), which is configured to execute the computer programs and/or software, and the aforementioned memory, in which the software and/or computer programs are stored.

The software and/or computer programs may be stored on an electronically readable data storage medium that is separate from the control computer 22 and/or the magnetic resonance apparatus. The control computer 22 accesses the electronically readable data medium by the storage medium being loaded therein.

The region 32 of the patient 15 that is to be searched and/or screened with respect to an abnormality is preferably not encompassed by the region 33 to be examined, in particular is outside of the region 33 to be examined of the patient 15. For example, if the region 33 to be examined of the patient 15 is an organ, then the region 32 located around the organ is searched and/or screened with respect to an abnormality in the medical image data. The region 32 that is screened and/or searched with respect to an abnormality in the medical image data may in this case be directly adjacent to the region 33 to be examined of the patient 15. Furthermore, the region 32 that is screened and/or searched with respect to an abnormality in the medical image data may also be spaced apart at a distance from the region 33 to be examined of the patient 15.

Medical image data, in particular magnetic resonance image data, that images the region 32 of the patient 15 that is outside of the region 33 to be examined of the patient 15, are provided in a first method step 100. The region 33 to be examined of the patient 15 has already been selected and/or specified on the basis of preliminary examination data that preceded the medical imaging examination, in particular the magnetic resonance examination, on the patient 15.

Providing the medical image data, in particular magnetic resonance image data, may in this case be done by an acquisition of the medical image data, in particular magnetic resonance image data, of the region 32 of the patient 15 that is outside of the region 33 to be examined of the patient 15. In this case, a planning and/or execution of the medical imaging measurement, in particular a magnetic resonance measurement, for acquiring the medical image data, in particular the magnetic resonance image data, of the region 32 of the patient 15 that is outside of the region 33 to be examined of the patient 15 can be performed automatically by the control computer 22. The planning and/or execution of the medical imaging measurement, in particular a magnetic resonance measurement, for acquiring the medical image data, in particular the magnetic resonance image data, can be performed as a background process by the control computer 22, such that the user, in particular a member of the medical operating staff, is not interrupted in his or her activity during the diagnostic imaging examination on the patient 15.

The acquisition of the medical image data, in particular the magnetic resonance image data, of the region 32 of the patient 15 that is outside of the region 33 to be examined of the patient 15 can be accomplished by an overview measurement or a localizer measurement. The overview measurement or localizer measurement is preferably produced for the region 32 of the patient 15 that is to be searched and/or screened with respect to an abnormality outside of the region 33 to be examined of the patient 15.

Alternatively or in addition, the medical image data, in particular the magnetic resonance image data, of the region 32 of the patient 15 that is outside of the region 33 to be examined of the patient 15 may be a whole-body scan of the patient 15. The whole-body scan of the patient 15 can likewise be acquired by an overview measurement or a localizer measurement.

The medical image data, in particular the magnetic resonance image data, of the region 32 of the patient 15 that is outside of the region 33 to be examined of the patient 15 is inferior in terms of image quality to an image quality of diagnostic image data of the region 33 to be examined of the patient 15. For example, the medical image data, in particular the magnetic resonance image data, of the overview measurement or the localizer measurement in this case typically exhibits a lower image quality, in particular a lower spatial resolution, in the acquired medical image data of the region 32 that is outside of the region 33 to be examined of the patient 15, than an image quality in the medical and/or diagnostic image data of the region 33 to be examined of the patient 15.

The medical image data, in particular the magnetic resonance image data, of the region 32 of the patient 15 that is outside of the region 33 to be examined of the patient 15 are preferably acquired during the diagnostic imaging examination for acquiring diagnostic image data of the region 33 to be examined of the patient 15. For example, the medical image data of the region 32 that is outside of the region 33 to be examined of the patient 15 can be acquired during a measurement pause between two imaging measurements or else during a planning phase for setting measurement parameters for a pending imaging measurement of the diagnostic imaging examination.

Alternatively or in addition, providing medical image data that images the region 32 of the patient 15 that is outside of the region 33 to be examined of the patient 15 may be combined with the acquisition of diagnostic image data that are acquired for the purpose of resolving diagnostic issues pertaining to the region 33 to be examined of the patient 15. For example, the region 33 to be examined of the patient 15 may be a kidney of the patient 15. The kidney of the patient 15 is therefore imaged in the acquired diagnostic image data and, for example, a region 32 that is outside of the region 33 to be examined of the patient 15 is also visualized or imaged in a border region of the acquired diagnostic image data. This border region may be the region 32 of the patient 15 that is outside of the region 33 to be examined of the patient 15, and may image or visualize a further organ of the patient 15, such as the liver of the patient 15.

In a further method step 101, the medical image data, in particular the magnetic resonance image data, for the region 32 of the patient 15 that is outside of the region 33 to be examined of the patient 15 are evaluated. The evaluation is preferably accomplished automatically and/or autonomously by the control computer 22. For this purpose, the control computer 22 has a self-learning algorithm that implements the automatic evaluation of the medical image data, in particular the magnetic resonance image data, of the region 32 of the patient 15 that is outside of the region 33 to be examined of the patient 15. In this case, the self-learning algorithm is based on training data that is derived from assessed abnormalities in already-available clinical findings.

Typically, the self-learning algorithm is based on a machine learning technique in which knowledge is generated from experience. The machine learning is realized by artificial neural networks. With the machine learning process, the self-learning algorithm is able to recognize patterns and rules in learning data and/or training data, in particular in assessed medical image data and the interpretation and/or assessment associated therewith. In this case, the self-learning algorithm can learn from examples and generalize these following termination of the learning phase.

Furthermore, the self-learning algorithm also takes into consideration in this process data of a course of a disease and/or of preliminary examinations and/or further medical data of the patient 15, for example already-acquired and evaluated medical and/or diagnostic image data of the patient 15, in the evaluation of the medical image data. The course of a disease of the patient 15 may be a history of one or more disorders of the patient 15. Preliminary examinations may for example also comprise non-imaging preliminary examinations of the patient 15 or else imaging examinations carried out using an imaging device that is different from the current imaging device. The further medical data of the patient 15 may also include information relating to blood values and/or circulation values of the patient 15. During the evaluation of the medical image data by means of the self-learning algorithm, this also permits a targeted search for abnormalities in medical image data that images or visualizes defined and/or targeted regions 32 of the patient. These defined and/or targeted regions 32 of the patient 15 are selected automatically and/or autonomously by the control computer 22 and/or the self-learning algorithm on the basis of the further medical data and/or of the course of the disease and/or of preliminary examinations, said defined and/or targeted regions 32 of the patient 15 being outside of the region 33 to be examined of the patient 15. If, for example, the preliminary examinations and/or a course of a disease and/or the further medical data point to a pulmonary disease of the patient 15, a region 32 in the medical image data that images the lung region of the patient 15 can be focused on in the evaluation of the medical image data. During the evaluation of the medical image data, the self-learning algorithm may also take into consideration in particular data that includes already-acquired medical image data of the patient 15.

In this method step 101 of the evaluation, the medical image data of the regions 32 of the patient 15 that are outside of the region 33 to be examined of the patient 15 is evaluated and/or searched with respect to an abnormality. In this process, the abnormalities in the medical image data may be identified by virtue of a change in color and/or a change in contrast in the evaluated medical image data. In particular, subregions containing the abnormalities in the evaluated medical image data stand out by virtue of a change in contrast and/or by virtue of a change in color from subregions that visualize an environment of the abnormalities, where the environment may, for example, have a uniform and/or constant color. For example, the abnormality may be a dark spot within an imaged organ of the patient 15.

Following method step 101 of the evaluation of the medical image data, a further method step 101 is performed. In this further method step 102, abnormality information of the medical image data for the region 32 of the patient 15 that is outside of the region 33 to be examined of the patient 15 is generated. The abnormality information is generated automatically and/or autonomously by means of the control computer 22 of the magnetic resonance device 10. The abnormality information includes information as to whether the region 32 of the patient 15 that is outside of the region 33 to be examined of the patient 15 has an abnormality.

Furthermore, the abnormality information may also include information for further assessment measures that should be initiated with respect to the identified abnormality. Further assessment measures of said type may be for example suggestions for further and/or additional medical imaging examinations for the region 32 of the patient 15 in which an abnormality has been detected and which is not encompassed by the region to be examined of the patient 15. In this case, the information for further assessment measures may also be parameter settings, a region to be examined, information relating to possible administrations of contrast agent, a suggestion for the medical imaging device by means of which the further and/or additional medical imaging examination should be performed to the best possible effect, etc. for the further and/or additional medical imaging examination.

Next, in a further method step 103, the abnormality information is presented at the output unit 24 of the user interface 23. In this case, the abnormality information is preferably visualized together with the displayed image data of the diagnostic image data of the region 33 to be examined of the patient 15. This enables all of the information that is of importance or relevance for the assessment to be provided in full for a user, in particular for a medical assessor of the diagnostic image data.

Although modifications and changes may be suggested by those skilled in the art, it is the intention of the Applicant to embody within the patent warranted hereon all changes and modifications as reasonably and properly come within the scope of the Applicant's contribution to the art.

1. A method for detecting abnormalities in medical image data of a region of a patient that is outside of a region to be examined of the patient, said method comprising:
providing a computer with medical image data that depicts a region of a patient that is outside of a region to be examined of the patient, said region to be examined of the patient being designated in the computer by preliminary examination data;
in said computer, automatically evaluating the medical image data for the region that is outside of the region to be examined of the patient;
from said evaluating, generating, in the computer, abnormality information of the medical image data for the region that is outside of the region to be examined of the patient; and
from said computer, causing said abnormality information to be visualized at a display screen in communication with said computer.

2. A method as claimed in claim 1 comprising providing said medical image data to said computer by operating a medical imaging apparatus from said computer in order to acquire said medical image data that depicts the region of the patient that is outside of the region to be examined of the patient.

3. A method as claimed in claim 2 comprising, from said computer, operating said medical imaging apparatus to acquire said medical image data of the region of the patient that is outside of the region to be examined of the patient during a diagnostic examination for acquiring diagnostic image data of the region to be examined of the patient.

4. A method as claimed in claim 2 comprising planning in said computer, a medical imaging procedure and operating the medical imaging apparatus, according to the planned medical imaging procedure, in order to acquire said medical image data of the region of the patient that is outside of the region to be examined of the patient.

5. A method as claimed in claim 2 comprising, from said computer, operating said medical imaging apparatus in order to execute a whole-body scan of the patient in order to acquire said medical data image data of the region of the patient that is outside of the region to be examined of the patient.

6. A method as claimed in claim 2 comprising, from said computer, operating said medical imaging apparatus in order to acquire said medical image data of the region of the patient that is outside of the region to be examined of the patient with an image quality that is inferior to diagnostic image data acquired from the region to be examined of the patient.

7. A method as claimed in claim 1 comprising automatically evaluating said medical image data for the region that is outside of the region to be examined of the patient by executing a self-learning algorithm in said computer.

8. A method as claimed in claim 7 comprising executing said self-learning algorithm based on training data representing assessed abnormalities identified in pre-existing medical image data.

9. A method as claimed in claim 1 comprising executing said self-learning algorithm in said computer based on at least one of data representing a course of a disease, preliminary examination data, and additional medical data of the patient.

10. A method as claimed in claim 7 comprising executing said self-learning algorithm in said computer dependent on previously-acquired medical image data from the patient.

11. A method as claimed in claim 1 comprising, from said computer, visualizing said abnormality information at said display screen in displayed images of diagnostic image data representing said region to be examined of the patient.

12. A method as claimed in claim 1 comprising generating said abnormality information in said computer so as to include further assessment measures to be taken with respect to an abnormality represented in said abnormality information.

13. A method as claimed in claim 12 wherein said further assessment measures include a suggestion for a further medical examination of a region of the patient that comprises said abnormality.

14. A medical imaging apparatus comprising:
an image data acquisition scanner;
a display monitor having a display screen;
computer provided with medical image data that depicts a region of a patient that is outside of a region to be examined of the patient, said region to be examined of the patient being designated in the computer by preliminary examination data;
said computer being configured to automatically evaluate the medical image data for the region that is outside of the region to be examined of the patient;
said computer being configured to generate, from said evaluation, abnormality information of the medical image data for the region that is outside of the region to be examined of the patient; and
said computer being configured to cause said abnormality information to be visualized at said display screen.

15. A medical imaging apparatus as claimed in claim 14 wherein said control unit is configured to execute a self-learning algorithm in order to evaluate said medical image data of the region of the patient that is outside of the region to be examined of the patient.

16. A non-transitory, computer-readable data storage medium encoded with programming instructions, said storage medium being loaded into a computer system of a medical imaging apparatus, and said programming instructions causing said computer system to:
receive medical image data that depicts a region of a patient that is outside of a region to be examined of the patient, said region to be examined of the patient being designated in the computer by preliminary examination data;
evaluate the medical image data for the region that is outside of the region to be examined of the patient;
from said evaluation, generate abnormality information of the medical image data for the region that is outside of the region to be examined of the patient; and
cause said abnormality information to be visualized at a display screen in communication with said computer.

17. A non-transitory, computer-readable data storage medium as claimed in claim 16 wherein said programming instructions cause said control computer to execute a self-learning algorithm in order to evaluate said medical image data of the region of the patient that is outside of the region to be examined of the patient.
